SEQUENCE LISTING (9 sequences)

SEQ ID NO: 1 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - protein, 205 aa, Homo sapiens
MTPPERLFLPRVCGTTLHLLLLGLLLVLLPGAQGLPGVGLTPSAAQTARQHPKMHLAHSNLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 3 - protein, 244 aa, Homo sapiens
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 4 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 5 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 6 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 7 - protein, 281 aa, Homo sapiens [sequence omitted]

SEQ ID NO: 8 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 9 - protein, 198 aa, Mus musculus
MEGEGVQPLDENLENGSRPRFKWKKTLRLVVSGIKGAGMLLCFIYVCLQLSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

(19) United States
(12) Patent Application Publication
(10) Pub. No.: US 2013/0224145 A1
(43) Pub. Date: Aug. 29, 2013

(54) COMPLEX HAVING TUMOR VACCINE EFFECT, AND USE THEREOF

(71) Applicant: Ji-Yang Wang, Kanagawa (JP)

(72) Inventor: Ji-Yang Wang, Kanagawa (JP)

(73) Assignee: RIKEN, Wako-shi, Saitama (JP)

(21) Appl. No.: 13/816,830

(22) PCT Filed: Aug. 12, 2011
(86) PCT No.: PCT/JP2011/068452

§ 371 Date: May 20, 2013

(30) Foreign Application Priority Data
Aug. 13, 2010 (JP) 2010-181394

(51) Int. Cl. A61K 39/00

(52) U.S. Cl. CPC A61K 39/0011
USPC 424/85.1; 435/325; 424/195.11; 424/192.1

(57) ABSTRACT

The present invention provides a tumor cell-soluble TNF family member molecule complex containing a tumor cell and an isolated soluble TNF family member molecule, wherein the soluble TNF family member molecule is bound on a surface of the tumor cell such that it binds to a receptor of the TNF family member expressed on a surface of a cell other than the tumor cell, and stimulates the cell other than the tumor cell via the receptor, and a composition and a tumor vaccine, each containing the complex.

### TECHNICAL FIELD

The present invention relates to a complex containing a tumor cell and an isolated soluble TNF family member molecule, and a composition, a pharmaceutical composition, a tumor vaccine and the like, which contain the complex.

### BACKGROUND ART

The history of a tumor vaccine therapy using an isolated tumor tissue or tumor cell is old, the results of the first animal test were reported in 1978 (non-patent documents 1, 2). Thereafter, the first clinical test was reported in 1993 (non-patent document 3), and a huge number of animal tests and clinical tests have been performed ever since (non-patent documents 4, 5). However, a clear treatment effect of a tumor vaccine therapy has not been observed to date.

Recently, there are a number of reports concluding that introduction of a gene such as cytokine and the like into a tumor cell increases an antitumor effect. However, clinical application has not been implemented due to the possibility of side effects caused by a virus vector used for increasing the gene transfer efficiency, and problems of complicated method requiring cell culture for a long term and the like (non-patent documents 4, 5).

It has been reported in 1997 that a tumor is rapidly rejected by forcibly expressing Fas ligand (to be also referred to as FasL in the present specification), which is a protein belonging to the TNF family (to be also referred to as TNF family member in the present specification), in the tumor cell (non-patent document 6). The present inventors have analyzed this rejection phenomenon in more detail and showed that a T cell-dependent acquired immunity to a tumor cell, which is inherently not rejected by being immunologically self, is established by expressing FasL in the tumor cell (non-patent documents 7-12). Moreover, the acquired immunity was suggested to recognize plural tumor specific antigens (non-patent document 9). In other words, it has been clarified that a tumor cell, which is a self cell, is eliminated as a foreign substance by own immune system. As mentioned above, however, the method utilizing an expression vector is hardly employed in actual clinical practice, since it takes time and is associated with the problem of side effects due to the vector.

Non-patent document 13 describes suppression of rejection of a pancreas graft by binding a streptavidin (SA)-FasL fusion protein to the pancreas with a biotinylated cell surface and inoculating same into an allo recipient. In addition, non-patent document 14 describes that rejection of vascular endothelial cell graft was suppressed by binding a streptavidin (SA)-FasL fusion protein to the vascular endothelial cell of allo with a biotinylated cell surface, and inoculating same into the recipient.

### DOCUMENT LIST

### Non-Patent Documents

non-patent document 1: Cancer Res. 38: 204-209 (1978).
non-patent document 2: Cancer Res. 39:1353-1360 (1979).
non-patent document 3: J. Clin. Oncol. 11: 390-9 (1993).
non-patent document 4: J. Clin. Invest. 114: 450-62 (2004).
non-patent document 5: Rev. Recent Clin. Trials 1: 283-92 (2006).
non-patent document 6: Nat. Med. 3: 165-70 (1997).
non-patent document 7: Int. J. Mol. Med. 9: 281-285 (2002).
non-patent document 8: Anticancer Res. 22: 831-836 (2002).
non-patent document 9: J. Immunol. 169: 2241-2245 (2002).
non-patent document 10: Cancer Gene Ther. 10: 134-140 (2003).
non-patent document 11: Clinical Immunology 39: 532-536 (2003)
non-patent document 12: Cancer Gene Ther. 14: 262-267 (2007).
non-patent document 13: Mol. Immunol. 44(11): 2884-2892 (2007)
non-patent document 14: Circulation 107:1525-1531 (2003)

### SUMMARY OF THE INVENTION

### Problems to be Solved by the Invention

From tumor cells generally having plural gene mutations and expressing various tumor antigens, a tumor vaccine using a peptide antigen and the like derived from a protein specifically expressed in a tumor cell can eliminate cells expressing the particular protein used as the antigen, but cannot eliminate other tumor cells. Moreover, in a conventional tumor treatment using a tumor tissue or tumor cell derived from the administration subject, to establish immunity to the tumor cell which is immunologically self, an expression vector that expresses a gene to recognize the tumor cell as non-self needs to be used.

Therefore, the present invention aims to provide a tumor vaccine that not only eliminates tumor cells expressing a particular tumor antigen, but also shows a superior therapeutic or prophylactic effect on a tumor existing in the body of an administration subject, without using an expression vector, and the like, and an active ingredient used therefor.

### Means of Solving the Problems

In view of the aforementioned problems, the present inventors have conducted intensive studies of a method for inducing immunity to various tumor antigens in a tumor cell by the binding of a substance having an adjuvanticity directly on the tumor cell surface without using an expression vector. As a result, when a tumor cell bound with SA-Fast on the cell surface was administered to a syngenic mouse, said tumor cell was rejected or its growth was suppressed, as in the case of expression of FasL using an expression vector. Furthermore, when the tumor cell was subjected to a fixation treatment and inoculated to an individual, immunity to the syngenic tumor cell was established and, when a tumor cell of the same line which was free of SA-FasL binding was administered again, this tumor cell was rejected and the growth was markedly suppressed. Surprisingly, this tumor vaccine effect was stronger than when FasL was expressed by using an expression vector. Based on these findings, the present inventors have further studied and completed the present invention.

Accordingly, the present invention provides the following.

[1] A tumor cell-soluble TNF family member molecule complex comprising a tumor cell and an isolated soluble TNF family member molecule, wherein
the soluble TNF family member molecule is bound on a surface of the tumor cell such that it binds to a receptor of the TNF family member expressed on a surface of a cell other than the tumor cell, and stimulates the cell other than the tumor cell via said receptor.
[2] The complex of [1], wherein the TNF family member is any selected from the group consisting of FasL, TNF, CD40L, OX40L, TRAIL and RANKL or a combination of two or more thereof.
[3] The complex of [2], wherein the TNF family member is FasL, OX40L, or a combination of FasL, TNF and OX40L.
[4] The complex of [1], wherein the soluble TNF family member molecule is multimerized.
[5] The complex of [1], wherein the soluble TNF family member molecule is a fusion molecule having an extracellular region of the TNF family member or a functional fragment thereof and a region having affinity for the molecule on the tumor cell surface.
[6] The complex of [5], wherein the region having affinity for the molecule on the tumor cell surface is a polypeptide residue having affinity for the molecule on the tumor cell surface.
[7] The complex of [6], wherein the polypeptide having affinity for the molecule on the tumor cell surface is avidin or streptavidin.
[8] The complex of [1], which is fixed.
[9] The complex of [8], wherein the fixation is achieved by an aldehyde treatment.
[10] The complex of [7], wherein the tumor cell is biotinylated.
[11] A composition comprising the complex of any of [1]-[10].
[12] The composition of [11] which is a medicament.
[13] A tumor vaccine comprising the complex of any of [1]-[10].
[14] The tumor vaccine of [13], wherein the tumor cell is isolated from an administration subject (individual).
[15] The tumor vaccine of [14], which is for a tumor treatment.
[16] A method of producing a tumor vaccine comprising the tumor cell-soluble TNF family member molecule complex of [1], comprising
(a) a step of mixing a tumor cell isolated from an administration subject (individual) and a soluble TNF family member molecule in an aqueous buffer,
(b) a step of obtaining a tumor cell-soluble TNF family member molecule complex by binding the soluble TNF family member molecule to a surface of the tumor cell in the mixture of (a), and
(c) a step of fixing the tumor cell-soluble TNF family member molecule complex obtained in (b).
[17] A method of treating or preventing a tumor, comprising administering, to a tumor patient or a patient having a clinical history of tumor, a tumor vaccine comprising a tumor cell-soluble TNF family member molecule complex comprising a tumor cell and an isolated soluble TNF family member molecule, wherein
the soluble TNF family member molecule is bound on a surface of the tumor cell such that it binds to a receptor of the TNF family member expressed on a surface of a cell other than the tumor cell, and stimulates the cell other than the tumor cell via said receptor.

### Effect of the Invention

Tumor can be effectively prevented or treated by using the complex, composition or tumor vaccine of the present invention. Particularly, by using a tumor cell isolated from an administration subject (individual) as a tumor cell contained in the complex of the present invention, immunity to a tumor cell, which is inherently immunologically self, is established, and then the tumor cell in the administration subject (individual) is eliminated. Therefore, when the complex, composition or tumor vaccine of the present invention is administered to a patient who received a treatment to remove the tumor by surgery and the like, the immunity to various kinds of tumor antigens specifically expressed in autologous tumor cells is activated, and tumor cells possibly remaining in the body of the patient (e.g., micrometastatic tumor cell) can be eliminated. Therefore, the complex, composition or tumor vaccine of the present invention is particularly effective for the prevention of recurrence of tumor after a tumor treatment.

When a protein is expressed on the cell surface using an expression vector, gene introduction by an expression vector takes a long time, and a high level expression of the object protein may not be possible due to (1) the introduction efficiency of the vector into the cell, (2) the promoter-enhancer activity of the vector in the cell, (3) the cytotoxicity of the protein to be expressed, and the like. However, the complex etc. of the present invention are not affected by the influence of such properties of the cell and molecule per se on the production steps, and can be stably produced in a very short time.

Therefore, the present invention can provide a rapid and stable treatment means and a rapid and stable recurrence-preventive means, which are effective for various tumors.

### BRIEF DESCRIPTION OF THE DRAWINGS

FIG. 1 shows that a streptavidin-FasL fusion protein shows strong cytotoxicity. Human T cells were cultured for 24 hr in the presence of 1 μg of streptavidin-FasL fusion protein or 1 μg of an anti-Fas antibody, and stained with Annexin V and Propidium Iodide (PI). Annexin V positive cell is an apoptotic cell, and PI positive cell is a necrotic cell after apoptosis. It has been shown that streptavidin-FasL fusion protein has a stronger activity to induce cell death than the anti-Fas antibody.

FIG. 2 shows binding of streptavidin-FasL fusion protein to the tumor cell surface. After biotinylation of the protein on the tumor cell surface, the streptavidin-FasL fusion protein was bound to the biotinylated protein, and the FasL expression level on the cell surface was analyzed by flow cytometry.

FIG. 3 shows suppression of the growth of tumor cell bound with FasL on the cell surface. Using A11 lung cancer cell (left) or B16 melanoma (right), post-inoculation growth was examined. The tumor cell bound with FasL on the cell surface was not rejected post-inoculation, but the growth decreased as compared to the control. The tumor cell made to express FasL by using a vector was rejected. The data shows mean±S.D. of 5 individuals.

FIG. 4 shows a vaccine effect on a tumor cell bound with FasL on the cell surface and fixed. Using a tumor cell bound with FasL on the cell surface and fixed or a tumor cell made to express FasL by using a vector was subcutaneously inoculated as a vaccine to a mouse, a control tumor cell was inoculated 2 weeks later, and the growth thereof was examined. The tumor cell bound with FasL on the cell surface and fixed showed a higher vaccine effect as compared to that by using vector. The data shows mean±S.D. of 5 individuals.

FIG. 5 shows a metastasis suppressive effect on a tumor cell bound with FasL on the cell surface and fixed. A tumor cell bound with FasL on the cell surface and fixed or a tumor cell made to express FasL by using a vector was subcutaneously inoculated as a vaccine to a mouse, a control tumor cell was inoculated 2 weeks later, and the metastasis to the lung was examined. The tumor cell bound with FasL on the cell surface and fixed and the cell made to express FasL by using a vector showed a higher metastasis suppressive effect as compared to the control. The data shows mean±S.D. of 5 individuals.

FIG. 6 shows a metastasis suppressive effect on A11 lung cancer cell bound with soluble TNF family member molecule on the cell surface and fixed. A wild-type A11 lung cancer cell was previously inoculated to a mouse and, on day 7 and day 21 post-inoculation, the A11 lung cancer cell bound with soluble TNF family member molecule and fixed or the control cell was subcutaneously inoculated. The metastasis to the lung was examined 29 days after the inoculation of the wild-type A11 lung cancer cell. The tumor cells bound with FasL alone, OX40L alone, or a combination of FasL, TNF and OX40L on the cell surface and fixed showed a higher metastasis suppressive effect as compared to the control cell. The data shows mean of 5 individuals.

### DESCRIPTION OF EMBODIMENTS

### 1. Tumor Cell-Soluble TNF Family Member Molecule Complex

The present invention provides a tumor cell-soluble TNF family member molecule complex comprising a tumor cell and an isolated soluble TNF family member molecule, wherein the soluble TNF family member molecule is bound on a surface of the tumor cell such that it binds to a receptor of the TNF family member expressed on a surface of a cell other than the tumor cell, and stimulates the cell other than the tumor cell via said receptor (hereinafter to be also referred to as the complex of the present invention).

### 1-1. Tumor Cell

As a tumor cell contained in the complex of the present invention, any tumor cell derived from a mammal, which is an administration subject of the tumor vaccine and the like of the present invention, can be used. Examples of the mammal include experiment animals such as rodents (e.g., mouse, rat, hamster, guinea pig and the like) and rabbit and the like, pets such as dog, cat and the like, domestic animals such as bovine, swine, goat, horse, sheep and the like, primates such as human, monkey, orangutan, chimpanzee and the like, and the like. While it is not particularly limited, preferred are rodents (mouse etc.) and primates (human etc.).

Examples of the tumor include solid tumor (e.g., epithelial tumor, nonepithelial tumor), and tumor in hematopoietic tissue. More particularly, examples of the solid tumor include, but are not limited to, gastrointestinal cancer (e.g., gastric cancer, colon cancer, colorectal cancer, rectal cancer), lung cancer (e.g., small cell cancer, non-small cell cancer), pancreatic cancer, kidney cancer, liver cancer, thymus cancer, spleen cancer, thyroid cancer, adrenal cancer, prostate cancer, urinary bladder cancer, ovarian cancer, uterine cancer (e.g., endometrial carcinoma, cervical cancer), bone cancer, skin cancer, sarcoma (e.g., Kaposi's sarcoma), melanoma, blastoma (e.g., neuroblastoma), glandular cancer, squamous cell carcinoma, non-squamous cell carcinoma, brain tumor and the like. Examples of the tumor in hematopoietic tissues include, but are not limited to, leukemia (e.g., acute myeloid leukemia (AML), chronic myeloid leukemia (CML), acute lymphocytic leukemia (ALL), chronic lymphocytic leukemia (CLL), adult T cell leukemia (ATL), myelodysplastic syndrome (MDS)), lymphoma (e.g., T lymphoma, B lymphoma, Hodgkin lymphoma), myeloma (multiple myeloma) and the like.

As the tumor cell, a tumor cell contained in a tumor tissue isolated from an individual having the above-mentioned tumor can be passaged and used, or used without passaging. When a tumor cell contained in a tumor tissue is used, the tumor tissue itself may be used, or the tumor tissue may be treated with a protease such as trypsin, collagenase, hyaluronidase, elastase, pronase and the like to disperse the cells, and the tumor cell may be isolated from the obtained cell suspension.

The tumor cell to be used in the present invention is preferably isolated or purified. The “isolation” and “purification” mean that an operation to remove substances other than the objective target substance has been applied. The purity of the tumor cell contained in the isolated or purified cell (ratio of tumor cell number relative to the total cell number) is generally not less than 30%, preferably not less than 50%, more preferably not less than 70%, more preferably not less than 80%, most preferably not less than 90% (e.g., 100%).

### 1-2. Soluble TNF Family Member Molecule

TNF family is a group of cytokines called tumor necrosis factors, which act on other cells by binding to a receptor and induce various responses. As TNF family members, a dozen or more proteins are known. While all of them can be used in the present invention, TNF family member is preferably selected from the group consisting of TNF (TNF-α, TNF-β or LT-β), FasL, CD40L, OX40L, TRAIL and RANKL.

The TNF family member used in the present invention is generally derived from a mammal. Examples of the mammal include experiment animals such as rodents (e.g., mouse, rat, hamster, guinea pig and the like) and rabbit and the like, pets such as dog, cat and the like, domestic animals such as bovine, swine, goat, horse, sheep and the like, primates such as human, monkey, orangutan, chimpanzee and the like, and the like. While the mammal is not particularly limited, preferred are rodents (mouse etc.) and primates (human etc.), and more preferred is human.

A TNF family member being derived from a mammal means that the amino acid sequence of the TNF family member is of the mammal.

The amino acid sequences of the TNF family members of major mammals are known, and available from the databases such as NCBI and the like. For example, the amino acid sequences and NCBI accession numbers of human TNF-α, TNF-β, LT-β, FasL, CD40L, OX40L, TRAIL, and RANKL are shown in Table 1.

TNF family member is generally expressed as a type II membrane protein in the body. In the present specification, an extracellular region of the TNF family member refers to a region on the C-terminal side from the transmembrane region. It is known that this C-terminal side region is cut off by an action of a specific protease, from the N terminal side region including the transmembrane region, released as a soluble form and functions. The extracellular regions and cleavage sites by protease of TNF-α, TNF-β, LT-β, FasL, CD40L, OX40L, TRAIL, and RANKL in human are shown in Table 1.

TABLE 1

| | | | | | region |
|---|---|---|---|---|---|
| | | | | cleavage | necessary |
| | | NCBI | extra- | site | for |
| gene | SEQ | accession | cellular | with | activity |
| name | ID NO | No. | domain | protease | expression |
| human | 1 | CAA25650 | 58-233 | 76/77 | 77-233 |
| TNF-α | | | | | |
| human | 2 | BAA00064 | 35-205 | unknown | 35-205 |
| TNF-β | | | | | |
| human | 3 | AAD18089 | 48-244 | none | 54-244 |
| LT-β | | | | | |
| human | 4 | AAC50071 | 103-281 | assumed | 137-281 |
| FasL | | | | to be | |
| | | | | near | |
| | | | | 127/128 | |
| human | 5 | NP_000065 | 47-261 | 112/113 | 113-261 |
| CD40L | | | | | |
| human | 6 | NP_003317 | 45-183 | unknown | 52-183 |
| OX40L | | | | | |
| human | 7 | NP_003801 | 39-281 | unknown | 95-281 |
| TRAIL | | | | | |
| human | 8 | NP_003692 | 71-317 | unknown | 136-317 |
| RANKL | | | | | |

The soluble TNF family member molecule contained in the complex of the present invention contains an extracellular region of at least any of the TNF family members or a functional fragment thereof.

The length of the functional fragment of an extracellular region of the TNF family member is not particularly limited as long as the soluble TNF family member molecule containing the fragment binds to a receptor corresponding to the TNF family member and shows an activity to stimulate, via said receptor, the cell expressing said receptor. It is generally not less than 130 amino acids, preferably not less than 140 amino acids, more preferably not less than 150 amino acids. The position of the functional fragment in the extracellular region is not particularly limited as long as the soluble TNF family member molecule containing the fragment binds to a receptor corresponding to the TNF family member and shows an activity to stimulate, via said receptor, the cell expressing said receptor. It is preferably the C-terminal thereof. One embodiment of the functional fragment in the extracellular region of the TNF family member is a region on the C-terminal side than the cleavage site by the aforementioned protease.

Preferable examples of the functional fragment in an extracellular region of the TNF family member include, but are not limited to, polypeptide consisting of the 77th-233rd amino acids of the amino acid sequence shown by SEQ ID NO: 1 for human TNF-α, polypeptide consisting of the 137th-281st amino acids of the amino acid sequence shown by SEQ ID NO: 4 for human FasL, polypeptide consisting of the 113th-261st amino acids of the amino acid sequence shown by SEQ ID NO: 5 for human CD40L, polypeptide consisting of the 52nd-183rd amino acids of the amino acid sequence shown by SEQ ID NO: 6 for human OX40L, polypeptide consisting of the 95th-281st amino acids of the amino acid sequence shown by SEQ ID NO: 7 for human TRAIL, polypeptide consisting of the 136th-317th amino acids of the amino acid sequence shown by SEQ ID NO: 8 for human RANKL, and the like.

The receptor of human TNF-α is human TNFR1, the receptor of human TNF-β is human TNFR1, the receptor of human LT-β is human LTβR, the receptor of human FasL is human Fas, the receptor of human CD40L is human CD40, the receptor of human OX40L is human OX40, the receptor of human TRAIL is human TRAIL-R1(DR4) or human TRAIL-R2(DR5), and the receptor of human RANKL is human RANK.

The soluble TNF family member molecule used in the present invention binds to a receptor corresponding to the TNF family member, and has an activity to stimulate a cell (preferably mammalian cell) expressing said receptor via said receptor.

Soluble TNF-α molecule binds to TNFR1 and has an activity to stimulate a cell (preferably mammalian cell) expressing TNFR1 via TNFR1. The stimulation by soluble TNF-α molecule specifically means induction of apoptosis.

Soluble TNF-β molecule binds to TNFR1 and has an activity to stimulate a cell (preferably mammalian cell) expressing TNFR1 via TNFR1. The stimulation by soluble TNF-β molecule specifically means induction of apoptosis.

Soluble LT-β molecule binds to LTβR and has an activity to stimulate a cell (preferably mammalian cell) expressing LTβR via LTβR. The stimulation by soluble LT-β molecule specifically means induction of apoptosis.

Soluble FasL molecule binds to Fas and has an activity to stimulate a cell (preferably mammalian cell) expressing Fas via Fas. The stimulation by soluble FasL molecule specifically means induction of apoptosis.

Soluble CD40L molecule binds to CD40 and has an activity to stimulate a cell (preferably mammalian cell) expressing CD40 via CD40. The stimulation by soluble CD40L molecule specifically means promotion of the growth of a cell (e.g., B cells).

Soluble OX40L molecule binds to OX40 and has an activity to stimulate a cell (preferably mammalian cell) expressing OX40 via OX40. The stimulation by soluble OX40L molecule specifically means promotion of the growth of a cell (e.g., CD4+T cells).

Soluble TRAIL molecule binds to DR4 or DR5 and has an activity to stimulate a cell (preferably mammalian cell) expressing DR4 or DR5 via DR4 or DR5. The stimulation by soluble TRAIL molecule specifically means induction of apoptosis.

Soluble RANKL molecule binds to RANK and has an activity to stimulate a cell (preferably mammalian cell) expressing RANK via RANK. The stimulation by soluble RANKL molecule specifically means cell death inhibitory activity or induction of differentiation.

The apoptosis-inducing activity can be evaluated by, for example, as described in the below-mentioned Example 2, culturing cells expressing a receptor of the corresponding TNF family member in a medium containing a soluble TNF family member molecule at a concentration of 1 μg/ml for 24 hr, staining the cultured cells with an antibody specific to Annexin V and propidium iodide, and analyzing the ratio of apoptotic cells (Annexin V positive, PI negative) by flow cytometry.

When the apoptosis-inducing activity of the soluble human FasL molecule is evaluated, for example, Jurkat cells expressing human Fas are preferably used. When the apoptosis-inducing activities of the soluble human TNF-α molecule, soluble human TNF-β molecule, soluble human LT-β molecule and soluble human TRAIL molecule are evaluated, for example, mouse L-929 cells expressing the receptors of these molecules are preferably used.

The cell growth promoting activity of soluble CD40L can be evaluated by, as described in, for example, WO2002/088186, culturing mammalian cell expressing CD40 for 3 days in a medium containing soluble CD40L at a concentration of 1 μg/ml, and measuring the cell growth by [³H]thymidine uptake. For example, when the cell growth promoting activity of soluble human CD40L molecule is evaluated, human tonsil B cells expressing human CD40 are preferably used.

The cell growth promoting activity of soluble OX40L can be evaluated by, as described in, for example, Mol. Immunol. 44, 3112-3121, 2007, culturing CD4+T cells for 2 days under an anti-CD3 antibody stimulation in a medium containing soluble OX40L at a concentration of 0.1 μg/ml, and measuring, by [³H]thymidine uptake, the promoted growth by the addition of soluble OX40L to CD4+T cells under anti-CD3 antibody stimulation. For example, when the cell growth promoting activity of soluble human OX40L molecule is evaluated, human activated CD4+T cells obtained by stimulating human peripheral CD4+T cells expressing human OX40, with IL-2 and PHA for 2 days are preferably used (Mol. Immunol. 44, 3112-3121, 2007).

The cell death inhibitory activity of soluble RANKL molecule can be evaluated by, as described in, for example, J. Exp. Med., 186:2075 (1997), culturing dendritic cells for 2 days in a medium containing soluble RANKL at a concentration of 1 μg/ml, and measuring, based on the ratio of surviving cell number, the inhibitory effect on the spontaneous cell death of the dendritic cells by the addition of soluble RANKL. When the cell death inhibitory activity of soluble human RANKL molecule is evaluated, dendritic cells derived from human peripheral CD14+monocyte, which can be obtained by the method described in J. Immunol. Methods 196: 121-135 (1996), are preferably used.

The TNF family member molecule contained in the complex of the present invention is soluble. The “soluble” means being capable of dissolving at 0.1 μg or more in 1 cc of purified water at 20° C.

The soluble TNF family member molecule contained in the complex of the present invention is an isolated or purified molecule. The “isolation” and “purification” mean that an operation to remove substances other than the objective target substance has been applied. The purity of the soluble TNF family member molecule contained in the isolated or purified soluble TNF family member molecule (weight ratio of soluble TNF family member molecule relative to the total protein weight) is generally not less than 30%, preferably not less than 50%, more preferably not less than 70%, more preferably not less than 80%, most preferably not less than 90% (e.g., 100%). Therefore, a soluble TNF family member molecule expressed by a gene present in a tumor cell contained in the complex of the present invention is not included in the “isolated or purified soluble TNF family member molecule”. That is, the soluble TNF family member molecule contained in the complex of the present invention is a molecule present outside the tumor cell and independently from the tumor cell before being contained in the complex.

The TNF family member molecule is generally known to show higher binding activity to the corresponding receptor when it is a multimer rather than a monomer, and shows stronger physiological activity as a result thereof. Thus, a soluble TNF family member molecule to be used in the present invention is preferably multimerized. Multimerization means association of two or more molecules. The number of molecules of the TNF family member molecule contained in the multimer of the TNF family member molecule is not limited as long as the TNF family member molecule binds to a receptor corresponding to the TNF family member and has an activity to stimulate, via said receptor, a cell that expresses said receptor, and it is generally 2-8, preferably 2-4. Whether or not the TNF family member molecule is multimerized, and the number of molecules of the TNF family member molecule contained in the multimer can be determined by dissolving the TNF family member molecule in an appropriate aqueous buffer and separating same by gel filtration.

The manner of multimerization is not limited as long as the obtained multimer of TNF family member molecule binds to a receptor corresponding to the TNF family member and has an activity to stimulate, via said receptor, a cell that expresses said receptor.

For example, it is known that the TNF family member in nature exists as a trimer on a cellular membrane and, as for TNF-α and the like, a soluble form generated by cleavage with a specific protease (TNF-α converting enzyme (TACE)) also exists as a trimer. In one embodiment, therefore, multimerization of a TNF family member molecule is based on the inherent multimerization ability that the TNF family member has.

In another embodiment, the soluble TNF family member molecule used in the present invention contains, in addition to an extracellular region of the TNF family member or a functional fragment thereof, a region having a multimerization ability (hereinafter to be referred to as multimerization region), and is multimerized based on the multimerization ability. As such multimerization region, compounds such as polypeptide, nucleic acid and the like having a multimerization ability can be utilized. The multimerization region is preferably a polypeptide having a multimerization ability. Examples of the polypeptide having a multimerization ability include, but are not limited to, leucine zipper (dimer), Coiled-Coil domain (dimer), collagen (trimer), haemagglutinin (trimer), ornithine transcarbamylase (trimer), avidin (tetramer), streptavidin (tetramer), dnaB helicase (hexamer), hemocyanin (hexamer), haemerythrin (octamer), ACRP30 or ACRP30-like protein (WO96/39429, WO 99/10492, WO 99/59618, WO 99/59619, WO 99/64629, WO 00/26363, WO 00/48625, WO 00/63376, WO 00/63377, WO 00/73446, WO 00/73448 or WO 01/32868), collectin family polypeptides such as apM1 (Maeda et al., Biochem. Biophys. Res. Comm. 221: 286-9, 1996), Clq (Sellar et al., Biochem. J. 274: 481-90, 1991) and Clq-like protein (WO 01/02565), polypeptide containing a “coiled-coil” domain (Kammerer R A, Matrix Biol 1997 March; 15(8-9):555-65; discussion 567-8, Lombardi & al., Biopolymers 1996; 40(5):495-504; http://mdl.ipc.pku.edu.cn/scop/data/scop.1.008.001.html) such as Carilage Matrix Protein (CMP) (Beck & al., 1996, J. Mol. Biol., 256, 909-923), stretch of collagen repeat (US2008003226) and the like. The polypeptide having a multimerization ability is preferably leucine zipper, avidin or streptavidin, more preferably avidin or streptavidin. Since avidin and streptavidin have a multimerization ability and an activity to bind to a surface of an biotinylated tumor cell in combination, it is preferably used for the present invention.

While the manner of linkage of an extracellular region of the TNF family member or a functional fragment thereof, and a multimerization region is not limited as long as the extracellular region of the TNF family member or a functional fragment thereof, and the multimerization region are linked in a manner free of separation when the complex of the present invention is formed, for example, the both are linked by a covalent bond such as peptide bond, disulfide bond and the like, or a non-covalent bond such as hydrogen bond, hydrophobic bond, ionic bond and the like. From the aspect of the stability of the bond, the both are preferably linked by a covalent bond (e.g., peptide bond). Those of ordinary skill in the art can appropriately link them in such binding manner.

When a polypeptide having a multimerization ability is used as a multimerization region, a soluble TNF family member molecule used in the present invention is preferably a fusion protein containing an extracellular region of the TNF family member or a functional fragment thereof and a polypeptide residue having a multimerization ability. While the position of the polypeptide residue having a multimerization ability in the fusion protein may be any of the N-terminal side and the C-terminal side of an extracellular region of the TNF family member or a functional fragment thereof, as long as the fusion protein binds to a receptor corresponding to the TNF family member and has an activity to stimulate, via said receptor, a cell that expresses said receptor, it is preferably the N-terminal side.

The distance between an extracellular region of the TNF family member or a functional fragment thereof and a polypeptide residue having a multimerization ability in the fusion protein is not particularly limited as long as the fusion protein binds to a receptor corresponding to the TNF family member and shows an activity to stimulate, via said receptor, the cell expressing said receptor. However, from the aspects of easy synthesis and stability of the protein, the distance is shorter the more preferable, and an extracellular region of the TNF family member or a functional fragment thereof and a polypeptide residue having a multimerization ability are preferably linked via, for example, a linker polypeptide residue consisting of about 1-100 amino acids (preferably about 1-50 amino acids, more preferably about 1-25 amino acids, still more preferably about 1-10 amino acids) or a bond. The amino acid sequence of the linker polypeptide residue is not particularly limited as long as the fusion protein binds to a receptor corresponding to the TNF family member and shows an activity to stimulate, via said receptor, the cell expressing said receptor.

As one example of a preferable fusion protein, a fusion protein containing avidin or streptavidin and an extracellular region of FasL, which can form a tetramer and is disclosed in U.S. Pat. No. 7,238,360, US2009074870, Circulation. 2003; 107: 1525-1531, Mol Immunol. 2007; 44(11): 2884-2892, and Immunity. 2002; 17: 795-808 (all of these are incorporated in the present specification by reference) can be mentioned. In addition, another preferable multimer of soluble FasL is disclosed in US2008003226 (incorporated in the present specification by reference).

The soluble TNF family member molecule contained in the complex of the present invention is bound on a surface of the tumor cell contained in the complex of the present invention, such that it binds to a receptor of the TNF family member expressed on a surface of a cell other than the tumor cell, and stimulates the cell other than the tumor cell via said receptor. In a preferable embodiment, therefore, the soluble TNF family member molecule contained in the complex of the present invention contains a region having affinity for the molecule on the tumor cell surface. Examples of the combination of the molecule on the tumor cell surface and a region having affinity for the molecule are shown in Table 2, but the examples are not limited thereto.

TABLE 2

| | | region having affinity for |
|---|---|---|
| | molecule on tumor cell | molecule on tumor cell |
| | surface | surface |
| 1 | cell membrane lipid | substance that binds to |
| | | cell membrane lipid |
| 1-(1) | GM3 glycolipid | wheat germ lectin |
| 1-(2) | sphingomyelin | lysenin |
| 1-(3) | GM1 glycolipid | cholera toxin subunit |
| 1-(4) | cellular membrane | FM4-64 |
| 1-(5) | cellular membrane | DiI |
| 2 | extracellular | substance that binds to |
| | polysaccharide | extracellular |
| | | polysaccharide (lectin |
| | | etc.) |
| 2-(1) | p-galactoside | galectin |
| 3 | specific antigen exposed | antibody specifically |
| | on cell surface | binding to the antigen |
| 4 | receptor protein | ligand specifically |
| | | binding to the receptor |
| | | protein |
| 5 | molecule introduced on | polypeptide specifically |
| | tumor cell surface by | binding to the molecule |
| | chemical modification | |
| 5-(1) | biotin | avidin or streptavidin |

The region having affinity for the molecule on the tumor cell surface is preferably a polypeptide having affinity for the molecule on the tumor cell surface. The polypeptide having affinity for the molecule on the tumor cell surface is preferably avidin or streptavidin. Since avidin and streptavidin have the above-mentioned multimerization ability and an activity to strongly bind to the surface of a biotinylated tumor cell in combination, they are preferably used for the present invention.

While the manner of linkage of an extracellular region of the TNF family member or a functional fragment thereof, and a region having affinity for the molecule on the tumor cell surface is not limited as long as the extracellular region of the TNF family member or a functional fragment thereof, and the region having affinity for the molecule on the tumor cell surface are linked in a manner free of separation when the complex of the present invention is formed and the soluble TNF family member molecule binds to a receptor of the TNF family member expressed on a surface of a cell other than the tumor cell, and stimulates the cell other than the tumor cell via said receptor while being bound on the surface of the tumor cell, for example, the both are linked by a covalent bond such as peptide bond, disulfide bond and the like, or a non-covalent bond such as hydrogen bond, hydrophobic bond, ionic bond and the like. From the aspect of the stability of the bond, the both are preferably linked by a covalent bond (e.g., peptide bond). Those of ordinary skill in the art can appropriately link them in such binding manner.

When the polypeptide having affinity for the molecule on the tumor cell surface is used as a region having affinity for the molecule on the tumor cell surface, the soluble TNF family member molecule used in the present invention is preferably a fusion protein containing an extracellular region of the TNF family member or a functional fragment thereof and the polypeptide residue having affinity for the molecule on the tumor cell surface. While the position of the polypeptide residue having affinity for the molecule on the tumor cell surface in the fusion protein may be any of the N-terminal side and C-terminal side of an extracellular region of the TNF family member or a functional fragment thereof as long as the fusion protein binds to a receptor of the TNF family member expressed on a surface of a cell other than the tumor cell, and stimulates the cell other than the tumor cell via said receptor while being bound on the surface of the tumor cell, it is preferably the N-terminal side.

The distance between an extracellular region of the TNF family member or a functional fragment thereof in the fusion protein and a polypeptide residue having affinity for the molecule on the tumor cell surface is not particularly limited as long as the fusion protein binds to a receptor of the TNF family member expressed on a surface of a cell other than the tumor cell, and stimulates the cell other than the tumor cell via said receptor while being bound on the surface of the tumor cell. However, from the aspects of easy synthesis and stability of the protein, the distance is shorter the more preferable, and an extracellular region of the TNF family member or a functional fragment thereof and a polypeptide residue having affinity for the molecule on the tumor cell surface are preferably linked via, for example, a linker polypeptide residue consisting of about 1-100 amino acids (preferably about 1-50 amino acids, more preferably about 1-25 amino acids, still more preferably about 1-10 amino acids) or a bond. The amino acid sequence of the linker polypeptide residue is not particularly limited as long as the fusion protein binds to a receptor of the TNF family member expressed on a surface of a cell other than the tumor cell, and stimulates the cell other than the tumor cell via said receptor while being bound on the surface of the tumor cell.

As one example of a preferable fusion protein, a fusion protein containing avidin or streptavidin and an extracellular region of FasL, which can form a tetramer and is disclosed in U.S. Pat. No. 7,238,360, US2009074870, Circulation. 2003; 107: 1525-1531, Mol Immunol. 2007; 44(11): 2884-2892, and Immunity. 2002; 17: 795-808 (all of these are incorporated in the present specification by reference) can be mentioned.

In a preferable embodiment, the soluble TNF family member molecule used in the present invention is a fusion protein containing an extracellular region of the TNF family member or a functional fragment thereof, a polypeptide residue having a multimerization ability, and a polypeptide residue having affinity for the molecule on the tumor cell surface. While the positional relationship of the three constitution factors in the fusion protein is not particularly limited as long as the fusion protein binds to a receptor of the TNF family member expressed on the surface of a cell other than the tumor cell, and can stimulate the cell other than the tumor cell via said receptor while being bound on the surface of the tumor cell, these constituent elements are preferably contained in the fusion protein in the order of from the N-terminal side, the polypeptide residue having affinity for the molecule on the tumor cell surface, the polypeptide residue having a multimerization ability, and the extracellular region of the TNF family member or a functional fragment thereof.

In this case, the distance between the polypeptide residue having a multimerization ability and the polypeptide residue having affinity for the molecule on the tumor cell surface is not particularly limited as long as the fusion protein binds to a receptor of the TNF family member expressed on a surface of a cell other than the tumor cell, and stimulates the cell other than the tumor cell via said receptor while being bound on the surface of the tumor cell. However, from the aspects of easy synthesis and stability of the protein, the distance is shorter the more preferable, and the polypeptide residue having a multimerization ability and the polypeptide residue having affinity for the molecule on the tumor cell surface are preferably linked via, for example, a linker polypeptide residue consisting of about 1-100 amino acids (preferably about 1-50 amino acids, more preferably about 1-25 amino acids, still more preferably about 1-10 amino acids) or a bond. The amino acid sequence of the linker polypeptide residue is not particularly limited as long as the fusion protein binds to a receptor of the TNF family member expressed on a surface of a cell other than the tumor cell, and stimulates the cell other than the tumor cell via said receptor while being bound on the surface of the tumor cell. In addition, the distance between the extracellular region of the TNF family member or a functional fragment thereof, and the polypeptide residue having a multimerization ability is as mentioned above.

As mentioned above, since avidin and streptavidin have multimerization ability and an activity to strongly bind to the surface of a biotinylated tumor cell in combination, one residue of avidin or streptavidin can perform the functions of both the polypeptide residue having a multimerization ability and the polypeptide residue having affinity for the molecule on the tumor cell surface. Therefore, in most preferable embodiments, the soluble TNF family member molecule to be used in the present invention is a fusion protein containing an extracellular region of the TNF family member or a functional fragment thereof, and a polypeptide residue of avidin or streptavidin. While the position of the residue of avidin or streptavidin in the fusion protein may be any of the N-terminal side and the C-terminal side of an extracellular region of the TNF family member or a functional fragment thereof, as long as the fusion protein binds to a receptor of the TNF family member expressed on the surface of a cell other than the tumor cell, and can stimulate the cell other than the tumor cell via said receptor while being bound on the surface of the tumor cell, it is preferably the N-terminal side. As one example of a preferable fusion protein, a fusion protein containing avidin or streptavidin and an extracellular region of FasL, which is disclosed in U.S. Pat. No. 7,238,360, US2009074870, Circulation. 2003; 107: 1525-1531, Mol Immunol. 2007; 44(11): 2884-2892, and Immunity. 2002; 17: 795-808 (all of these are incorporated in the present specification by reference) can be mentioned.

The soluble TNF family member molecule used in the present invention may contain any amino acid sequence in the N-terminal and/or the C-terminal thereof as long as it binds to a receptor of the TNF family member expressed on a surface of a cell other than the tumor cell, and can stimulate the cell other than the tumor cell via said receptor while being bound on the surface of the tumor cell. As such amino acid sequence, an affinity tag for the purification or detection of soluble TNF family member molecule can be mentioned. Such affinity tag is well known in the field and, for example, FLAG peptide (Hopp et al., Biotechnology 6: 1204 (1988)), c-Myc tag, His tag, Fc tag, HA tag and the like can be recited.

The soluble TNF family member molecule can be obtained by various known methods. For example, it can be obtained by a method for isolating and purifying from a tissue, cell or culture supernatant of a mammal such as human, rat, mouse etc. that naturally express a TNF family member molecule according to a method known per se or a method analogous thereto; a method for chemically synthesizing according to a peptide synthesis method known per se using a peptide.synthesizer and the like; a method for producing and cultivating a transformant containing DNA encoding a soluble TNF family member molecule; a method for biochemically synthesizing using a nucleic acid encoding a soluble TNF family member molecule as a template and a cell-free transcription/translation system, or the like.

When the above-mentioned polypeptide is prepared from a tissue or cell of a mammal naturally expressing a TNF family member molecule, it can be isolated and purified by homogenizing the tissue or cell, extracting same with acid, alcohol or the like, and subjecting the extract to a protein separation technique known per se (e.g., salting out, dialysis, gel filtration, chromatography such as reversed-phase chromatography, ion exchange chromatography, affinity chromatography etc., and the like). In addition, it can also be isolated and purified from a culture supernatant in the same manner.

A soluble TNF family member molecule can be chemically synthesized using a commercially available peptide.synthesizer.

When the above-mentioned polypeptide is prepared using a transformant containing DNA, the polypeptide can be produced by obtaining a polynucleotide encoding a soluble TNF family member molecule, transforming the host with an expression vector containing the polynucleotide, and cultivating the obtained transformant. For example, refer to the methods described in Molecular Cloning, 2nd ed.; J. Sambrook et al., Cold Spring Harbor Lab. Press (1989) and the like. A polynucleotide encoding a soluble TNF family member molecule can be produced by linking a polynucleotide encoding each of the aforementioned constituent elements (extracellular region of the TNF family member or a functional fragment thereof, polypeptide residue having a multimerization ability and polypeptide residue having affinity for the molecule on the tumor cell surface) constituting a soluble TNF family member molecule by using an appropriate enzyme such as ligase and the like by a known gene recombination technique. A polynucleotide encoding each constituent element constituting a soluble TNF family member molecule can be directly amplified by PCR by utilizing each known sequence information and sequence information described in the Sequence Listing in the present specification to design a suitable primer and using a DNA clone encoding each constituent element and the like as a template. Alternatively, a polynucleotide encoding each constituent element may be synthesized based on the sequence information and using a polynucleotide synthesizer.

The obtained polynucleotide can be used as it is according to the object, or after digesting with a restriction enzyme or adding a linker when desired. The polynucleotide has ATG as a translation initiation codon at the 5′-terminal side, and may have TAA, TGA or TAG as a translation stop codon at the 3′-terminal side. In addition, a signal sequence of a secreted protein (e.g., IL-4 etc.) may be added to the 5′-terminal to efficiently secrete a soluble TNF family member molecule extracellularly. Such signal sequence is well known to those of ordinary skill in the art and can be appropriately selected by those of ordinary skill in the art. These translation initiation codon, translation stop codon and signal sequence can be added using a suitable synthetic DNA adapter.

An expression vector capable of expressing a soluble TNF family member molecule can be produced by functionally linking the obtained polynucleotide to the downstream of a promoter in a suitable expression vector. The kind of the expression vector can be appropriately determined according to a host to be used. A transformant introduced with said vector can be produced by introducing the expression vector into a host by a gene transfer method known per se (e.g., lipofection method, calcium phosphate method, microinjection method, protoplast fusion method, electroporation method, DEAE dextran method, gene transfer method by Gene Gun etc.). A soluble TNF family member molecule can be produced by culturing the transformant by a method known per se according to the kind of the host, and isolating or purifying the soluble TNF family member molecule from the culture.

When a cell-free transcription/translation system is utilized, a method including, in the same manner as above, synthesizing mRNA using an expression vector inserted with a DNA encoding the above-mentioned polypeptide prepared by a known cloning method (e.g., expression vector wherein the DNA is under the control of T7 or SP6 promoter etc., and the like) as a template and a translation reaction solution containing an RNA polymerase and a substrate (NTPs) matching the promoter, and performing a translation reaction using the mRNA as a template and a known cell-free translation system (e.g., Escherichia coli, rabbit reticulocyte, extract of wheat germ etc.) and the like can be mentioned.

### 1-3. Binding of Soluble TNF Family Member Molecule and Tumor Cell

In the complex of the present invention, the soluble TNF family member molecule is bound on a surface of the tumor cell such that it binds to a receptor of the TNF family member expressed on a surface of a cell other than the tumor cell, and stimulates the cell other than the tumor cell via said receptor.

In the present specification, the complex means a substance obtained by covalent binding or non-covalent binding of plural constituent factors.

Whether a soluble TNF family member molecule can bind to a receptor of the TNF family member expressed on a surface of a cell other than the tumor cell, and stimulates the cell other than the tumor cell via said receptor while being bound on the surface of the tumor cell can be evaluated by using a complex containing a tumor cell and a soluble TNF family member molecule in the test for evaluation of the activity of soluble TNF family member molecule described in detail in the above-mentioned (1-2. Soluble TNF family member molecule), instead of the soluble TNF family member molecule. In this evaluation, a complex containing a tumor cell and a soluble TNF family member molecule, and a cell expressing a receptor of the TNF family member are mixed at, for example, 1:1 cell number ratio.

The tumor cell and soluble TNF family member molecule are bound such that the coupling constant is at least 10⁷M, preferably 10⁹M or more, more preferably 10¹²M or more. Such binding can be selected from covalent bonds such as peptide bond, disulfide bond and the like, or non-covalent bonds such as hydrogen bond, hydrophobic bond and the like.

As mentioned above, when a soluble TNF family member molecule contained in the complex of the present invention contains a region having affinity for the molecule on the surface of the tumor cell contained in the complex of the present invention, the region having affinity contained in the soluble TNF family member molecule is bound to the molecule on the tumor cell surface, whereby the soluble TNF family member molecule is bound on the tumor cell surface. Examples of the combination of the molecule on the tumor cell surface and a region having affinity for the molecule are shown in Table 2.

While the amount of the soluble TNF family member molecule contained in the complex of the present invention is not limited as long as the complex can stimulate, via said receptor, a cell expressing a receptor of the corresponding TNF family member, 1,000-100,000, preferably 5,000-50,000, molecules of the soluble TNF family member are generally bound on the cell surface of one tumor cell.

The complex of the present invention can contain 2 or more kinds of different soluble TNF family member molecules. For example, 2 or more kinds (for example, 2-5 kinds, preferably 2 or 3 kinds) of soluble TNF family member molecules, including each different extracellular region of the TNF family member or a functional fragment thereof, are bound to one tumor cell. When two kinds of soluble TNF family member molecules are used, examples of the combination of the TNF family members include FasL+TNF-α; FasL+TNF-β; FasL+LT-β; FasL+CD40L; FasL+OX40L; FasL+TRAIL; FasL+RANKL; TNF-α+TNF-β; TNF-α+LT-β; TNF-α+CD40L; TNF-α+OX40L; TNF-α+TRAIL; TNF-α+RANKL; TNF-β+LT-β; TNF-β+CD40L; TNF-β+OX40L; TNF-β+TRAIL; TNF-β+RANKL; LT-β+CD40L; LT-β+OX40L; LT-β+TRAIL; LT-β+RANKL; CD40L+OX40L; CD40L+TRAIL; CD40L+RANKL; OX40L+TRAIL; OX40L+RANKL; TRAIL+RANKL and the like. When three kinds of soluble TNF family member molecules are used, examples of the combination of the TNF family members include a combination of any 3 kinds of soluble TNF family member molecules selected from the group consisting of TNF-α, TNF-β, LT-β, FasL, CD40L, OX40L, TRAIL and RANKL. FasL+TNF-α+OX40L is preferable since it shows a high metastasis suppressive effect as shown in the below-mentioned Example 6. Respective soluble TNF family member molecules may be bound to a tumor cell in different manners or the same manner. From the aspect of efficient production of the complex of the present invention, the same manner is preferable. For example, in one embodiment, all soluble TNF family member molecules contained in the complex of the present invention are fusion proteins containing an extracellular region of the TNF family member or a functional fragment thereof, and a polypeptide residue of avidin or streptavidin, wherein the fusion protein is bound to biotin on the cell surface of the tumor contained in the complex. When two or more kinds of different soluble TNF family member molecules are used in combination, the amount of each soluble TNF family member molecule contained in the complex of the present invention is not particularly limited as long as the complex can stimulate, via said receptor, a cell expressing a receptor of the corresponding TNF family member. When two or more kinds of different soluble TNF family member molecules are used in combination, cells expressing receptors of different TNF family members can be stimulated simultaneously. Therefore, many kinds of cells can be acted on than using only one kind of soluble TNF family member molecule. When two or more kinds of different soluble TNF family member molecules are used in combination, superior effects such as (1) reduction of dose, (2) sustained treatment effect, (3) synergistic effect, and the like can be expected as compared to the use of only one kind of soluble TNF family member molecule.

The complex of the present invention can be produced by mixing tumor cell and soluble TNF family member molecule in an aqueous buffer, and binding the soluble TNF family member molecule to the surface of the tumor cell. Here, examples of the aqueous buffer include, but are not limited to, a culture supernatant of a host cell used in production of the soluble TNF family member molecule, a cell culture medium for cultivating tumor cell, phosphate buffer or the like.

While the mixing ratio of the tumor cell and the soluble TNF family member molecule is not limited as long as the obtained complex of the present invention can stimulate a cell expressing a receptor of the corresponding TNF family member via said receptor, for example, 1,000 or more, 5,000 or more, 10,000 or more, 50,000 or more, preferably 100,000 or more, soluble TNF family member molecules are mixed per one tumor cell.

A tumor cell in a living state or dead state is used for the production of the complex of the present invention. A living tumor cell includes a tumor tissue isolated from an individual, a tumor cell isolated from a tumor tissue, a tumor cell obtained by passage of such tumor tissue or tumor cell, passaged or unpassaged tumor tissue or tumor cell after a radiation treatment and the like. A dead tumor cell includes a fixed tumor tissue or tumor cell, a cryopreserved tumor tissue or tumor cell and the like.

When a radiation treatment is performed, a dose sufficient and appropriate for preventing the growth of tumor cells is selected according to the kind of the tumor cell and the condition of the sample. For example, a dose of 20-40 Gy can be irradiated on a tumor cell having high radiation sensitivity, and a does of 70 Gy or more can be irradiated on a tumor cell having low sensitivity. The radiation includes X-RAY, β-ray, baryon beam, proton beam and the like.

A tumor cell can be fixed by a known method using an aldehyde type fixative such as formaldehyde, para-formaldehyde, glutaraldehyde and the like, a fixative containing an acid such as picric acid, tannic acid, osmic acid and the like, a fixative containing a metal salt such as mercuric chloride, zinc acetate, zinc chloride, zinc sulfate and the like, a dehydrating agent type fixative such as methanol, ethanol, acetone and the like. Of these, an aldehyde treatment using an aldehyde type fixative is preferable, particularly preferably para-formaldehyde, to maintain the steric structure of an antigen contained in the tumor cell. A method of fixing such tumor cell is well known to those of ordinary skill in the art.

When cryopreservation is performed, cells are suspended in a solvent such as a medium and the like, dispensed to a freeze resistant container, and frozen at 0° C. or less, preferably −20° C. or less, particularly preferably −70° C. or less. In addition, when a tissue containing a tumor cell, for example, a tissue isolated from an individual is fixed by freezing, the tissue is placed in a freeze resistant container, and frozen together with an embedding material for freezing or as it was. Freezing can be performed using a commercially available apparatus such as a rapid freezing apparatus, a high pressure freezing apparatus and the like.

When the soluble TNF family member molecule contained in the complex of the present invention contains a region having affinity for the molecule on the surface of the tumor cell contained in the complex of the present invention, the region having affinity and contained in the soluble TNF family member molecule is bound to the molecule on the tumor cell surface, whereby the soluble TNF family member molecule is bound on the tumor cell surface.

When avidin or streptavidin is used as a region having affinity for the molecule on the tumor cell surface, the surface of the tumor cell is preferably biotinylated in advance. The surface of the tumor cell can be biotinylated by treating the surface of the tumor cell with, for example, a biotinylating reagent such as Sulfo-NHS-biotin (manufactured by Pierce) and the like.

When the soluble TNF family member molecule contained in the complex of the present invention does not contain a region having affinity for the molecule on the surface of the tumor cell contained in the complex of the present invention, a soluble TNF family member molecule can be bound to the tumor cell surface by using a method such as chemical crosslinking and the like. For example, a soluble TNF family member molecule can be covalently bound to the tumor cell surface by using a photoreactive crosslinking agent such as Sulfo-NHS-Diazirine (manufactured by Pierce) and the like.

A soluble TNF family member molecule can be bound to the tumor cell surface by a means such as the above-mentioned biotin-streptavidin interaction, chemical crosslinking and the like, according to the protocol provided by the manufacturer of a reagent to be used, or under reaction conditions known per se. Particularly, since an interaction between biotin and streptavidin is extremely strong, utilizing such interaction, a tumor cell-soluble TNF family member molecule complex can be obtained by, for example, gently stirring by a rotator and the like at 4° C. for 5 min.

After binding a soluble TNF family member molecule to the surface of a tumor cell, the obtained complex is washed with an appropriate buffer and the like, and unreacted soluble TNF family member molecule is removed, whereby the complex of the present invention can be isolated or purified. The purity of the complex contained in the isolated or purified complex of the present invention (ratio of protein weight of the complex relative to the total protein weight) is generally 30% or more, preferably 50% or more, more preferably 70% or more, further preferably 80% or more, most preferably 90% or more (e.g., 100%).

The complex of the present invention is preferably fixed. This is because a tumor vaccine effect is enhanced by fixing the complex of the present invention. The complex of the present invention can be fixed by a known method using an aldehyde type fixative such as formaldehyde, para-formaldehyde, glutaraldehyde and the like, a fixative containing an acid such as picric acid, tannic acid, osmic acid and the like, a fixative containing a metal salt such as mercuric chloride, zinc acetate, zinc chloride, zinc sulfate and the like, a dehydrating agent type fixative such as methanol, ethanol, acetone and the like. Of these, an aldehyde treatment using an aldehyde type fixative is preferable, particularly preferably para-formaldehyde, to maintain the steric structure of an antigen contained in the tumor cell. A method of fixing such tumor cell is well known to those of ordinary skill in the art.

### 2. Use of the Complex of the Present Invention

Using the complex of the present invention, a specific immune response to a tumor cell can be activated. Therefore, by administering the complex of the present invention as a tumor vaccine, an immune response to a tumor cell of a patient can be activated, the tumor can be treated or prevented, metastasis of the tumor cell can be suppressed or recurrence of the tumor can be prevented.

While the kind of the tumor is not particularly limited, for example, it is a solid tumor (e.g., epithelial tumor, nonepithelial tumor) or a tumor in a hematopoietic tissue. More particularly, examples of the solid tumor include gastrointestinal cancer (e.g., gastric cancer, colon cancer, colorectal cancer, rectal cancer), lung cancer (e.g., small cell cancer, non-small cell cancer), pancreatic cancer, kidney cancer, liver cancer, thymus cancer, spleen cancer, thyroid cancer, adrenal cancer, prostate cancer, urinary bladder cancer, ovarian cancer, uterine cancer (e.g., endometrial carcinoma, cervical cancer), bone cancer, skin cancer, sarcoma (e.g., Kaposi's sarcoma), melanoma, blastoma (e.g., neuroblastoma), glandular cancer, squamous cell carcinoma, non-squamous cell carcinoma, brain tumor and the like. Examples of the tumor in hematopoietic tissues include leukemia (e.g., acute myeloid leukemia (AML), chronic myeloid leukemia (CML), acute lymphocytic leukemia (ALL), chronic lymphocytic leukemia (CLL), adult T cell leukemia (ATL), myelodysplastic syndrome (MDS)), lymphoma (e.g., T lymphoma, B lymphoma, Hodgkin lymphoma), myeloma (multiple myeloma) and the like.

When the complex of the present invention is used as a tumor vaccine, it can be formulated as a pharmaceutical composition according to a conventional means. The complex of the present invention is low toxic, and can be administered directly as a liquid or a pharmaceutical composition in a suitable dosage form, to a mammal (e.g., rat, rabbit, sheep, swine, bovine, cat, dog, monkey, human etc.) orally or parenterally (e.g., intravascular administration, subcutaneous administration etc.). The complex of the present invention is generally administered parenterally.

In a preferable embodiment, the tumor cell contained in the complex of the present invention is separated from an individual to be the administration subject. For example, a part of a tumor tissue or tumor cell of a tumor patient is separated by a surgery or biopsy from the patient, and the complex of the present invention is produced using same. This complex is then administered to the same tumor patient, whereby a specific immune response to the tumor cell is activated. This immune response attacks the tumor cell in the patient, whereby the tumor in the patient shrinks or disappears, or metastasis of the tumor cell to other part can be suppressed. Whether or not the patient has a tumor, and shrink or disappearance of the tumor can be determined by a method known in the field, for example, image diagnoses such as endoscopy, X-ray inspection, CT inspection, ultrasonography, MRI inspection and the like, cytological diagnosis, blood inspection, palpation and the like.

In another preferable embodiment, the complex of the present invention containing a tumor cell separated from a patient, for whom the tumor is judged to have shrunk or disappeared by a treatment such as surgery, chemical therapy, radiation therapy and the like (patient having a clinical history of tumor), is administered to the patient before treatment or during the treatment process. Such administration activates a specific immune response to the tumor cell and can eliminate the tumor cell possibly remaining in the body of the patient (e.g., micrometastasis), and therefore, the recurrence of the treatment target tumor in the same site or nearby site in the body can be suppressed, and metastasis and growth of the tumor cell to and in other site can be suppressed.

As the tumor vaccine of the present invention, the complex of the present invention itself, which is the active ingredient thereof, may be administered or may be administered in the form of a suitable pharmaceutical composition. Examples of the pharmaceutical composition to be used for administration include those containing the above-mentioned complex of the present invention and a pharmaceutically acceptable carrier. As the pharmaceutically acceptable carrier, excipient, binder, lubricant, solvent, disintegrant, solubilizing agents, suspending agent, emulsifier, isotonicity agent, stabilizer, soothing agent, preservative, antioxidant, corrigent, colorant and the like can be added. Examples of the excipient include organic excipients such as saccharides (lactose, glucose, D-mannitol etc.), celluloses (starches, crystalline cellulose etc.) and the like, and inorganic excipients such as calcium carbonate, kaolin etc., and the like. Examples of the binder include pregelatinized starch, gelatin, gum arabic, methylcellulose, carboxymethylcellulose, sodium carboxymethylcellulose, crystalline cellulose, D-mannitol, trehalose, hydroxypropylcellulose, hydroxypropylmethylcellulose, polyvinylpyrrolidone, polyvinyl alcohol and the like. Examples of the lubricant include stearic acid, fatty acid salt such as stearate and the like, talc, silicates and the like. Examples of the solvent include purified water, physiological brine and the like. Examples of the disintegrant include low-substituted hydroxypropylcellulose, chemically-modified cellulose and starches and the like. Examples of the solubilizing agent include polyethylene glycol, propylene glycol, trehalose, benzyl benzoate, ethanol, sodium carbonate, sodium citrate, sodium salicylate, sodium acetate and the like. Examples of the suspending agent or emulsifier include sodium lauryl sulfate, gum arabic, gelatin, lecithin, glycerol monostearate, polyvinyl alcohol, polyvinylpyrrolidone, celluloses such as sodium carboxymethylcellulose and the like, polysorbates, polyoxyethylene hydrogenated castor oil and the like. Examples of the isotonicity agent include sodium chloride, potassium chloride, saccharides, glycerol, urea and the like. Examples of the stabilizer include polyethylene glycol, dextran sodium sulfate, other amino acids and the like. Examples of the soothing agent include glucose, calcium gluconate, procaine hydrochloride and the like. Examples of the preservative include paraoxybenzoates, chlorobutanol, benzyl alcohol, phenethyl alcohol, dehydroacetic acid, sorbic acid and the like. Examples of the antioxidant include sulfite, ascorbic acid and the like. Examples of the corrigent include sweetener, flavor and the like generally used in the pharmaceutical field and the like. Examples of the colorant include colorants generally used in the pharmaceutical field and the like. Such pharmaceutical composition is provided as a dosage form suitable for oral or parenteral administration.

A preparation preferable for oral administration includes liquid obtained by dissolving a component in a diluent such as water and saline, capsule, granule, powder, tablet containing a component as a solid or granule, suspension obtained by suspending a component in a suitable dispersing medium, emulsion obtained by dispersing a solution containing a component, in a suitable dispersing medium and emulsifying the dispersion, and the like.

Examples of the preparation preferable for parenteral administration (e.g., intravenous injection, subcutaneous injection, muscle injection, topical injection etc.) include aqueous and non-aqueous isotonic aseptic injection liquids. They may contain antioxidant, buffer, bacteriostatic, isotonicity agent and the like. In addition, it includes aqueous and non-aqueous aseptic suspensions, and they may contain suspending agent, solubilizer, thickener, stabilizer, preservative and the like. These preparations can be filled in a container by unit dose or plural doses such as ampoule and vial. Furthermore, the active ingredient and a pharmaceutically acceptable carrier may be freeze dried and preserved in a state for dissolution or suspending in a suitable aseptic vehicle immediately before use.

In addition, the pharmaceutical composition of the present invention may further contain, besides the complex of the present invention and the above-mentioned other components, an active ingredient according to the object of use. Examples of the active ingredient include adjuvant and the like. Examples of the adjuvant include, but are not limited to, precipitated adjuvants such as sodium hydroxide, aluminum hydroxide, aluminum phosphate, carboxylvinyl polymer and the like, incomplete Freund's adjuvant which is a mixture of paraffin and Arlacel, oil adjuvants such as complete Freund's adjuvant obtained by adding dead cells such as dead bacteria, Mycobacterium tuberculosis etc. to the incomplete Freund's adjuvant and the like.

The pharmaceutical composition contains an effective amount of the complex of the present invention. The content of the complex of the present invention in the pharmaceutical composition is generally about 0.1-100 wt %, preferably about 1-99 wt %, more preferably about 10-90 wt %, of the whole pharmaceutical composition.

The contents disclosed in any publication cited herein, including patents and patent applications, are hereby incorporated in their entireties by reference, to the extent that they have been disclosed herein.

The present invention is explained in more detail in the following by referring to Examples, which are not to be construed as limitative. Unless specifically indicated, the reagents, apparatuses and materials to be used in the present invention are commercially available.

### EXAMPLES

### Example 1

### Preparation of Streptavidin-FasL Fusion Protein (SAFasL)

(1) Genome DNA was extracted from actinomycetes (available from Incorporated Administrative Agency National Institute of Technology and Evaluation, Biotechnology Development Center), and a gene encoding streptavidin (SA) was amplified by the PCR method and isolated. On the other hand, messenger RNA was extracted from Jurkat T cell line (available from American Type Culture Collection (ATCC)), and cDNA was prepared using the extract as a template. Using the PCR method, a cDNA region encoding an extracellular region of human FasL and a cDNA region encoding a signal peptide of human interleukin 4 (IL-4) were amplified and isolated.
(2) The amplified DNA fragment was linked using the gene recombination technology, and a chimeric gene encoding secretion type SAFasL, wherein IL-4 signal peptide, SA and FasL extracellular region are linked in this order from the N-terminal, was constructed.
(3) An expression vector obtained by inserting the constructed chimeric gene into (pcDNA3, Invitrogen) was transiently introduced into 293T cells (obtained from ATCC). The cells were cultured in a DMEM medium containing 10% fetal bovine serum (Sigma Ltd.) under culture conditions of 5% CO₂ and 37° C. for 3 days, and the culture supernatant was recovered.
(4) SAFasL secreted in the culture supernatant was confirmed by Western blot.

### Example 2

### In Vitro Cytotoxicity of SAFasL

The cytotoxicity of the secreted SAFasL was evaluated using a human Jurkat T cell line. The Jurkat T cell is known to express Fas on the cell surface and, when bound with a FasL or anti-Fas antibody, undergo cell death via apoptosis. To Jurkat T cells (1 ml, 2.5×10⁵ cells) were added the culture supernatant obtained in Example 1 and containing 30 μl SAFasL or an anti-Fas antibody (obtained from BD Biosciences) to a concentration of 1 μg/ml, and the cells were cultured for 24 hr and stained with Annexin V, which is an apoptosis marker (horizontal axis), and Propidium Iodide that stains dead cells (vertical axis). As shown in FIG. 1, when the anti-Fas antibody was added, apoptosis cells were 29% and dead cells were 28%. When SAFasL was added, the dead cells reached 87%. That is, 30 μl of the supernatant was found to have cytotoxicity 3 times stronger than that of 1 μg of the anti-Fas antibody.

### Example 3

### Binding of SAFasL to Tumor Cell Surface

Using a commercially available biotinylating reagent (Sulfo-NHS-Biotin, PIERCE) according to the protocol of the manufacturer, the surface protein of tumor cell A11 (provided by Dr. Takenaga of the Chiba Cancer Center) was biotinylated.

The biotinylated cells (1×10⁶ cells) and the SAFasL-containing culture supernatant obtained in Example 1 (5 ml) were mixed. The mixture was reacted at 4° C. for 5 min while stirring with a rotator, and the cells were washed 3 times with 10 ml of saline (PBS) containing 100 mM Glycine to remove unreacted SAFasL.

The A11 tumor cells bound with SAFasL were stained with an anti-FasL antibody (BD Biosciences), and the expression of FasL was confirmed by FACS.

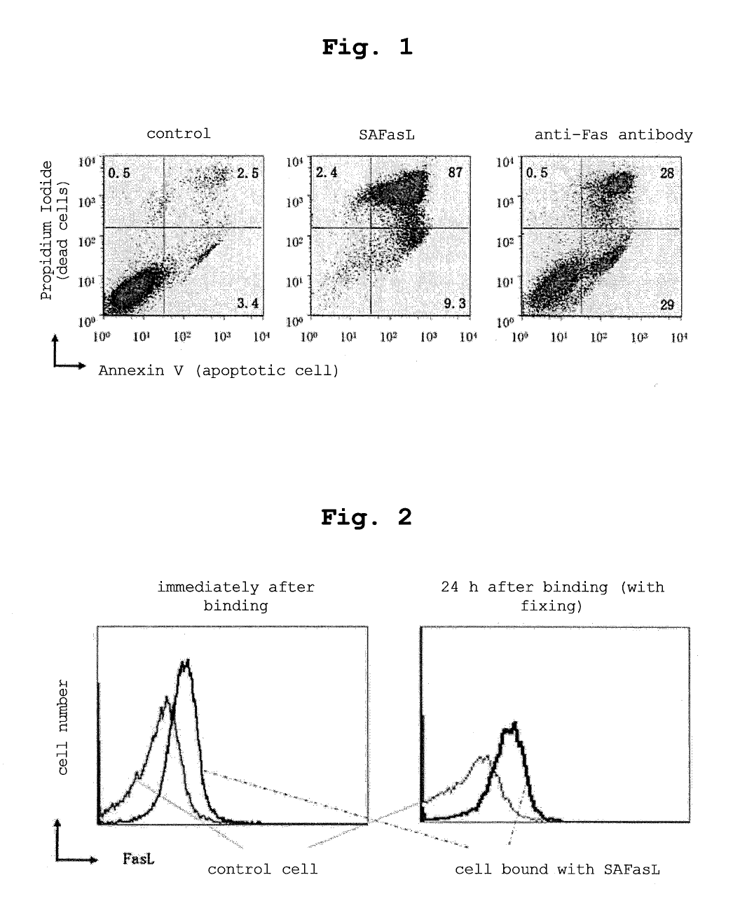

As shown in FIG. 2, left, SAFasL was found to have expressed at a high level.

SAFasL was bound to the cells, and the cells were fixed with 1% para-formaldehyde for 10 min while gently stirring by a rotator at room temperature and washed 3 times with 10 ml PBS. The abundance of SAFasL on the cell surface was measured in the same manner as above. As a result, expression of SAFasL on the cell surface at the same level as that immediately after binding was found even 24 hours after the fixing (FIG. 2, right).

### Example 4

### Suppression of Growth after Inoculation of Tumor Cell with SAFasL Bound on Cell Surface

A tumor cell made to express FasL by using an expression vector is immunologically self, but known to be rejected immediately after inoculation (Int. J. Mol. Med. 9: 281-285 (2002)). Therefore, whether a tumor cell bound with SAFasL is rejected like a tumor cell made to express FasL by using an expression vector was examined using A11 lung cancer cell or B16 melanoma cell. Both these tumor cells are of B6 mouse origin.

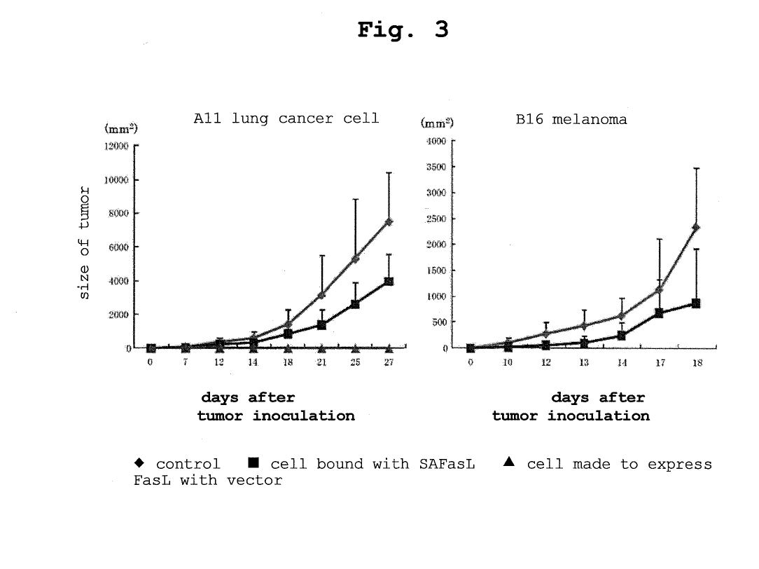

An A11 cell bound with SAFasL on the cell surface was produced by the method described in Example 3 (though without fixation). An A11 cell made to express FasL by using an expression vector was prepared as described in Int. J. Mol. Med. 9: 281-285 (2002). A control cell, a cell bound with SAFasL on the cell surface or a cell made to express FasL were subcutaneously inoculated by 2×10⁵ cells to B6 mice, and the size of the tumors was measured over time after inoculation. As shown in FIG. 3, left, the A11 cell made to express FasL by using a vector could not grow after inoculation and was rejected. This is the same result as that obtained before (Int. J. Mol. Med. 9: 281-285 (2002)). The A11 cell bound with SAFasL was not rejected, but the growth thereof was slow as compared to the control cell.

Similar results were also obtained in an experiment using B16 melanoma cells (obtained from ATCC) (FIG. 3, right). That is, SAFasL bound to the tumor exerted a certain level of growth suppressive effect on the tumor cells.

This result demonstrates the usefulness of the tumor cell bound with SAFasL on the cell surface.

### Example 5

### Vaccine Effect on Growth of and Metastasis Suppressive Effect on Wild-Type A11 Cell of A11 Lung Cancer Cell Bound with SAFasL and Fixed

As described in Example 4, when a tumor cell is not fixed, the growth of the tumor cell is not completely suppressed by binding SAFasL. On the other hand, when the tumor cell is fixed, it loses proliferative capacity. Therefore, the vaccine effect and the metastasis suppressive effect of a tumor cell bound with SAFasL on the cell surface and fixed were verified.

The same cells as in Example 4 were used for the test. The cells were fixed by the method described in Example 3. The A11 cell bound with 2×10⁵ SAFasL and fixed, A11 cell made to express FasL by using an expression vector, or saline as a control was subcutaneously inoculated to B6 mice as a vaccine. Two weeks later, 2×10⁵ wild-type A11 cells were subcutaneously inoculated, and the growth of the tumor and metastasis thereof to the lung were examined.

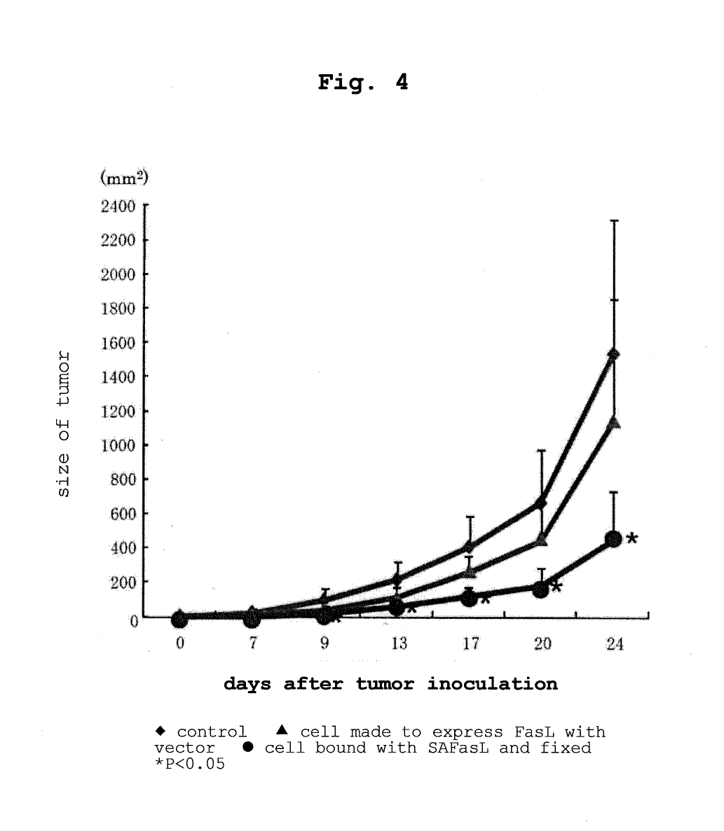

As shown in FIG. 4, the growth of the wild-type A11 cell was suppressed by using any of the A11 cell bound with SAFasL and fixed, and A11 cell made to express FasL by using an expression vector. This result shows that administration of a tumor cell bound with soluble FasL on the cell surface and fixed induces an immune response to syngenic tumor cells in the same manner as with the use of a tumor cell made to express FasL by using an expression vector, and the immune response leads to the rejection of syngenic tumor cells including tumor cell without expressing FasL.

Surprisingly, moreover, as shown in FIG. 4, the A11 cell bound with SAFasL and fixed showed a stronger antitumor effect than the A11 cell made to express FasL by using an expression vector. When the tumor cell was alive, use of the tumor cell made to express FasL by introducing an expression vector more strongly suppressed tumor growth than the tumor cell bound with SAFasL (Example 4). However, when the tumor cells were fixed, the tumor cell bound with SAFasL showed a stronger antitumor vaccine effect than the tumor cell made to express FasL by introducing an expression vector. This result shows that fixing a tumor cell bound with soluble FasL enhances the antitumor vaccine effect.

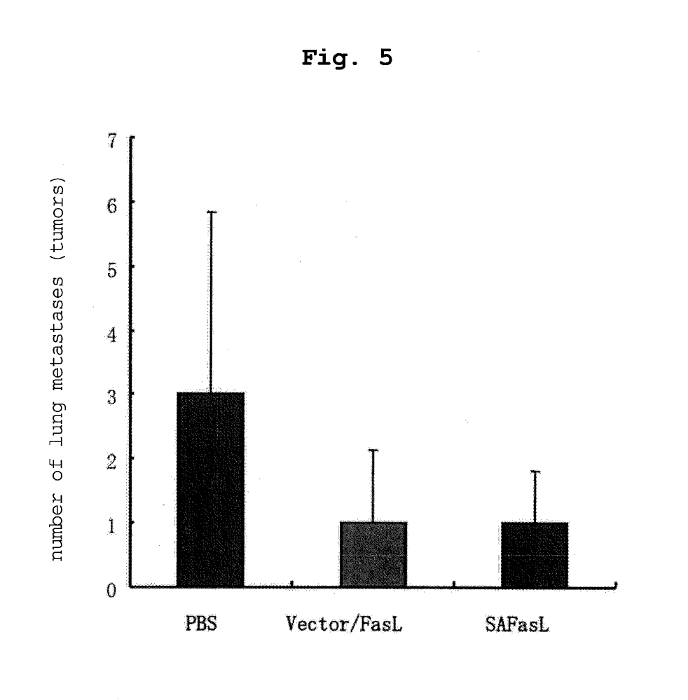

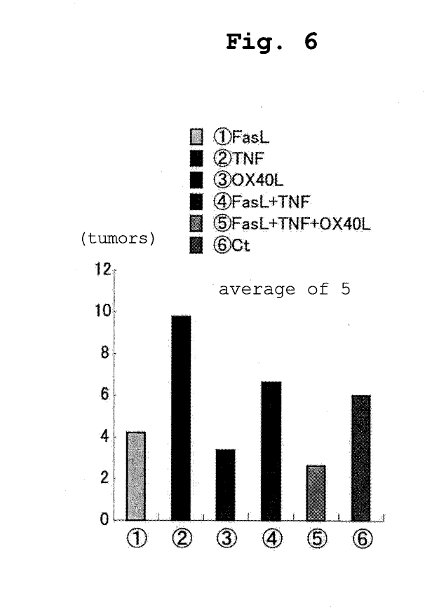

As shown in FIG. 5, the metastasis of the wild-type A11 cell to the lung was suppressed by using any of the A11 cell bound with SAFasL and fixed, and A11 cell made to express FasL by using an expression vector. This result shows that the tumor cell bound with SA-FasL and fixed has a high tumor metastasis suppressive effect equivalent to that of the tumor cell made to express FasL.

### Example 6

### Suppressive Effect of A11 Lung Cancer Cell Bound with Soluble TNF Family Member Molecule and Fixed on Metastasis of Wild-Type A11 Lung Cancer Cell

A metastasis suppressive effect of a tumor cell for an individual already having a solid tumor or tumor cell in the body is one of the important properties for clinical application of a tumor vaccine. Therefore, a suppressive effect of A11 lung cancer cell bound with a soluble TNF family member molecule and fixed on lung metastasis of lung cancer tumor cell in an individual having a solid tumor formed by inoculation of wild-type A11 lung cancer cell in advance was examined.

By a method similar to that in Example 1, streptavidin-FasL fusion protein (SAFasL), streptavidin-TNF fusion protein (SATNF) and streptavidin-OX40L fusion protein (SAOX40L) were produced by using an extracellular region of human FasL, an extracellular region of human TNF-α and an extracellular region (51-198) of mouse OX40L (NCBI accession No.: NP_{—}033478 (SEQ ID NO: 9)), respectively.

The A11 lung cancer cells bound with TNF family member molecule and fixed were produced by a method similar to that in Example 3 by using (1) SAFasL, (2) SATNF, (3) SAOX40L, (4) SAFasL+SATNF (1:1 (molar ratio)) and (5) SAFasL+SATNF+SAOX40L (1:1:1 (molar ratio)). In addition, A11 lung cancer cell treated in the same manner by using (6) control supernatant (DMEM medium containing 10% fetal bovine serum) was used as a control cell.

The lung metastasis suppressive effect was examined by the following method. The wild-type A11 lung cancer cells (2×10⁵) were subcutaneously inoculated to B6 mouse. Seven days later, the A11 lung cancer cell bound with a soluble TNF family member molecule and fixed, or control cell (2×10⁵ cells) was subcutaneously inoculated. 21 days after the inoculation of the wild-type A11 lung cancer cells, the A11 lung cancer cell bound with the soluble TNF family member molecule and fixed or the control cell (2×10⁵ cells), which was the same as that inoculated 7 days later, was subcutaneously inoculated again. 29 days after the inoculation of the wild-type A11 lung cancer cells, the lung was removed and tumors were counted.

As shown in FIG. 6, when the tumor cell bound solely with SAFasL or OX40L on the cell surface was inoculated, the number of lung tumors was smaller than that with the control cell. When the tumor cell bound with a combination of SAFasL, SATNF and SAOX40L on the cell surface was inoculated, the number of lung tumors was still smaller. From these results, it was shown that a particularly high tumor metastasis suppressive effect can be obtained when the TNF family member is FasL alone, OX40L alone, or a combination of FasL, TNF-α and OX40L. Since the present experiment system does not permit easy expression of a tumor metastasis suppressive effect, even when a tumor metastasis suppressive effect is not confirmed in the present experiment system, it does not deny the clinical effectiveness of the tumor cell bound with a soluble TNF family member molecule used. On the other hand, since the above-mentioned tumor metastasis suppressive effect was confirmed, the complex of the present invention (particularly, a complex wherein the TNF family member is FasL alone, OX40L alone, or a combination of FasL, TNF-α and OX40L) is strongly suggested to show a tumor metastasis suppressive effect also in the clinical practice.

### INDUSTRIAL APPLICABILITY

Since the complex of the present invention can be prepared rapidly and stably, an effective therapeutic agent and recurrence preventive agent for various tumors can be rapidly provided to tumor patients. In addition, the complex of the present invention enables production of such tumor vaccine.

The contents disclosed in any publication cited herein, including patents and patent applications, are hereby incorporated in their entireties by reference, to the extent that they have been disclosed herein.

This application is based on a patent application No. 2010-181394 filed in Japan (filing date: Aug. 13, 2010), the contents of which are incorporated in full herein.

1. A tumor cell-soluble TNF family member molecule complex comprising a tumor cell and an isolated soluble TNF family member molecule, wherein the soluble TNF family member molecule is bound on a surface of the tumor cell such that it binds to a receptor of the TNF family member expressed on a surface of a cell other than the tumor cell, and stimulates the cell other than the tumor cell via said receptor.

2. The complex according to claim 1, wherein the TNF family member is any selected from the group consisting of FasL, TNF, CD40L, OX40L, TRAIL and RANKL or a combination of two or more thereof.

3. The complex according to claim 2, wherein the TNF family member is FasL, OX40L, or a combination of FasL, TNF and OX40L.

4. The complex according to claim 1, wherein the soluble TNF family member molecule is multimerized.

5. The complex according to claim 1, wherein the soluble TNF family member molecule is a fusion molecule having an extracellular region of the TNF family member or a functional fragment thereof and a region having affinity for the molecule on the tumor cell surface.

6. The complex according to claim 5, wherein the region having affinity for the molecule on the tumor cell surface is a polypeptide residue having affinity for the molecule on the tumor cell surface.

7. The complex according to claim 6, wherein the polypeptide having affinity for the molecule on the tumor cell surface is avidin or streptavidin.

8. The complex according to claim 1, which is fixed.

9. The complex according to claim 8, wherein the fixation is achieved by an aldehyde treatment.

10. The complex according to claim 7, wherein the tumor cell is biotinylated.

11. A composition comprising the complex according to claim 1.

12. The composition according to claim 11, which is a medicament.

13. A tumor vaccine comprising the complex according to claim 1.

14. The tumor vaccine according to claim 13, wherein the tumor cell is isolated from an administration subject (individual).

15. The tumor vaccine according to claim 14, which is for a tumor treatment.

16. A method of producing a tumor vaccine comprising the tumor cell-soluble TNF family member molecule complex according to claim 1, comprising
(a) a step of mixing a tumor cell isolated from an administration subject (individual) and a soluble TNF family member molecule in an aqueous buffer,
(b) a step of obtaining a tumor cell-soluble TNF family member molecule complex by binding the soluble TNF family member molecule to a surface of the tumor cell in the mixture of (a), and
(c) a step of fixing the tumor cell-soluble TNF family member molecule complex obtained in (b).

17. A method of treating or preventing a tumor, comprising administering, to a tumor patient or a patient having a clinical history of tumor, a tumor vaccine comprising a tumor cell-soluble TNF family member molecule complex comprising a tumor cell and an isolated soluble TNF family member molecule, wherein the soluble TNF family member molecule is bound on a surface of the tumor cell such that it binds to a receptor of the TNF family member expressed on a surface of a cell other than the tumor cell, and stimulates the cell other than the tumor cell via said receptor.

18. The method according to claim 17, wherein the TNF family member is any selected from the group consisting of FasL, TNF, CD40L, OX40L, TRAIL and RANKL or a combination of two or more thereof.

19. The method according to claim 18, wherein the TNF family member is FasL, OX40L, or a combination of FasL, and TNF and OX40L.

20. The method according to claim 17, wherein the soluble TNF family member molecule is multimerized.

21. The method according to claim 17, wherein the soluble TNF family member molecule is a fusion molecule having an extracellular region of the TNF family member or a functional fragment thereof and a region having affinity for the molecule on the tumor cell surface.

22. The method according to claim 21, wherein the region having affinity for the molecule on the tumor cell surface is a polypeptide residue having affinity for the molecule on the tumor cell surface.

23. The method according to claim 22, wherein the polypeptide having affinity for the molecule on the tumor cell surface is avidin or streptavidin.

24. The method according to claim 17, wherein the complex is fixed.

25. The method according to claim 24, wherein the fixation is achieved by an aldehyde treatment.

26. The method according to claim 23, wherein the tumor cell is biotinylated.
